SEQUENCE LISTING (14 sequences)

SEQ ID NO: 1 - protein, 9 aa, Mus musculus
QQWSSNPWT

SEQ ID NO: 2 - protein, 9 aa, synthetic construct
  REGION 1..9: note = Synthetic Construct
QQGYGNPFT

SEQ ID NO: 3 - protein, 9 aa, Mus musculus
QQWSSNPYT

SEQ ID NO: 4 - protein, 9 aa, Mus musculus
QQGINNPLT

SEQ ID NO: 5 - protein, 9 aa, Mus musculus
QQWSFNPPT

SEQ ID NO: 6 - protein, 17 aa, Mus musculus
EINPTSGGTNFNEKFKS

SEQ ID NO: 7 - protein, 9 aa, synthetic construct
  REGION 1..9: note = Synthetic Construct
QQGYGNPFT

SEQ ID NO: 8 - protein, 9 aa, unidentified
  REGION 1..9: note = Synthetic Construct
QQWSSNPFT

SEQ ID NO: 9 - protein, 9 aa, unidentified
  REGION 1..9: note = Synthetic Construct
QHHSDNPWT

SEQ ID NO: 10 - protein, 9 aa, unidentified
  REGION 1..9: note = Synthetic Construct
QQWTSNPPT

SEQ ID NO: 11 - protein, 9 aa, unidentified
  REGION 1..9: note = Synthetic Construct
QQWTSNPPT

SEQ ID NO: 12 - protein, 9 aa, unidentified
  REGION 1..9: note = Synthetic Construct
QQWSSNPFT

SEQ ID NO: 13 - protein, 10 aa, unidentified
  REGION 1..10: note = Synthetic Construct
QQTYSNPPIT

SEQ ID NO: 14 - protein, 9 aa, unidentified
  REGION 1..9: note = Synthetic Construct
QQWSSNPPT

(19) United States
(12) Patent Application Publication
(10) Pub. No.: US 2023/0120018 A1
(43) Pub. Date: Apr. 20, 2023

(54) PREDICTION OF PEPTIDE CLEAVAGE IN POLYPEPTIDES THROUGH PHYSICS-BASED SIMULATIONS

(71) Applicant: Genentech, Inc., South San Francisco, CA (US)

(72) Inventors: Karthikan Rajagopal, South San Francisco, CA (US); Nisana Marie Andersen, South San Francisco, CA (US); Saeed Izadi, South San Franscisco, CA (US); Robert Franklin Kelley, JR., South San Francisco, CA (US)

(73) Assignee: Genentech, Inc., San Francisco, CA (US)

(21) Appl. No.: 18/058,110

(22) Filed: Nov. 22, 2022

Related U.S. Application Data
(63) Continuation of application No. PCT/US2021/041289, filed on Jul. 12, 2021.
(60) Provisional application No. 63/051,166, filed on Jul. 13, 2020.

(51) Int. Cl. G16B 15/20; G16B 40/20; G06N 7/01

(52) U.S. Cl. CPC G16B 15/20; G16B 40/20; G06N 7/01

(57) ABSTRACT

The present disclosure relates to polypeptide degradation, and in particular to techniques for predicting the likelihood that a peptide bond for a given polypeptide molecule is susceptible to a cleavage reaction. Particularly, aspects of the present disclosure are directed to generating a representation of a polypeptide, performing a molecular-dynamics simulation using the representation to obtain a set of polypeptide conformations, determining, for each polypeptide conformation, a spatial characteristic of an amino acid, estimating a nucleophilic attack distance of each polypeptide conformation based on the spatial characteristic, identifying a reactive conformation that is susceptible to a cleavage reaction based on the nucleophilic attack distance of each polypeptide conformation, determining a free energy of the spatial characteristic of the amino acid in the reactive conformation; and predicting a probability of the side chain of the amino acid being trapped in the reactive conformation based on the free energy.

### PRIORITY CLAIM

This application is a continuation of International Application No. PCT/US2021/041289, filed on Jul. 12, 2021, which claims the benefit of and priority to U.S. Provisional Application No. 63/051,166, filed on Jul. 13, 2020, which is hereby incorporated by reference in its entirety for all purposes.

### SEQUENCE LISTING

The official copy of the sequence listing is submitted electronically via Patent Center as an XML formatted sequence listing with a file named 1360159_Sequence_Listing.xml, created on Nov. 22, 2022, and having a size of 15.9 KB, and is filed concurrently with the specification. The sequence listing contained in this xml formatted document is part of the specification and is herein incorporated by reference in its entirety.

### FIELD

The present disclosure relates to polypeptide degradation, and in particular to techniques for predicting the likelihood that a peptide bond for a given polypeptide molecule is susceptible to a cleavage reaction.

### BACKGROUND

Polypeptide therapeutics have been successful and now represent a significant fraction of new drug approvals. In part this success can be attributed to the high affinity and specificity that can be achieved for polypeptides such as monoclonal antibody (mAb) against important disease targets. In addition, mAbs can have long serum half-life through interactions of the fragment crystallizable region (Fc region) (the tail region of an antibody) with an Fc region recycling receptor (FcRn) thus enabling less frequent dosing. In some disease settings, for example an acute treatment where long half-life is undesireable or in a tissue environment such as ocular where FcRn recycling is not active, an antigen-binding fragment (Fab) may be preferred over the intact mAb.

Despite these advantages as therapeutic agents, mAbs and antibody fragments can be susceptible to chemical and physical instability that can lead to degradation of the polypeptides and ultimately limit their utility. Physical instability may manifest as soluble aggregation, precipitation, and gel formation. Chemical instability may manifest as deamidation (e.g., asparagine (Asn) deamidation), isomerization (e.g., aspartic acid (Asp) isomerization), and oxidation (e.g., oxidation of tryptophan (Trp) and methionine (Met) residues), to name a few. In the context of biologics, degradation may reduce availability of a polypeptide therapeutic and/or reduce likelihood of triggering a target biological effect. For example, Asp isomerization can result in a loss of potency of the polypeptide therapeutic and isoaspartate formation from Asp isomerization has been linked to Alzheimer's disease. It would be advantageous to be able to detect the likelihood of degradation for a given polypeptide early during the therapeutic agent development process.

### SUMMARY

In various embodiments, a computer-implemented method is provided that includes determining, for a polypeptide conformation of a polypeptide comprising an amino acid having a side chain and a backbone, a dihedral angle for the backbone and a dihedral angle for the side chain of the amino acid while in the polypeptide conformation; determining a nucleophilic attack distance between two atoms, functional groups, or a combination thereof of the amino acid while in the polypeptide conformation based on the dihedral angle for the backbone and the dihedral angle for the side chain, where one of the two atoms or functional groups is in the side chain of the amino acid and another of the two atoms or functional groups is in the backbone of the amino acid; determining, based on the nucleophilic attack distance of the amino acid while in the polypeptide conformation, that the polypeptide conformation is a reactive conformation that is susceptible to a cleavage reaction; in response to determining the polypeptide conformation is the reactive conformation, determining a free energy of the dihedral angle for the backbone and the dihedral angle for the side chain of the amino acid while in the reactive conformation; and predicting a probability of the side chain of the amino acid being trapped in the reactive conformation based on the free energy of the dihedral angle for the backbone and the dihedral angle for the side chain of the amino acid.

In some embodiments, the computer-implemented method further comprises generating a representation of the polypeptide; and performing a molecular-dynamics simulation using the representation, wherein a result of the performance of the molecular-dynamics simulation comprises a set of polypeptide conformations for the polypeptide including the polypeptide conformation

In some embodiments, the computer-implemented method further comprises predicting a probability of the polypeptide to chemically degrade as a result of the side chain of the amino acid being trapped in the reactive conformation.

In some embodiments, the computer-implemented method further comprises outputting the probability of the side chain of the amino acid being trapped in the at least one reactive conformation and/or the probability of the polypeptide chemically degrading.

In some embodiments, the computer-implemented method further comprises removing the polypeptide from a list of potential polypeptides to be used as at least part of a therapeutic agent based on the probability of the side chain of the amino acid being trapped in the at least one reactive conformation and/or the probability of the polypeptide chemically degrading.

In some embodiments, the computer-implemented method further comprises ranking the polypeptide lower than another polypeptide in a list of potential polypeptides to be used as at least part of a therapeutic agent based on the probability of the side chain of the amino acid being trapped in the at least one reactive conformation and/or the probability of the polypeptide chemically degrading, wherein the probability of the side chain of the amino acid being trapped in the at least one reactive conformation and/or the probability of the polypeptide chemically degrading for the another polypeptide is less than the probability of the side chain of the amino acid being trapped in the at least one reactive conformation and/or the probability of the polypeptide chemically degrading for the polypeptide.

In some embodiments, the predicting the probability of the polypeptide to chemically degrade includes: identifying an accessibility constraint that, when satisfied, indicates that an amide group of the polypeptide has above-threshold spatial accessibility to bind with a solvent molecule from a surrounding solvent; and determining, for the reactive conformation, that the accessibility constraint is satisfied based on assessing one or more spatial characteristics of the polypeptide.

In some embodiments, the determining that the polypeptide conformation is the reactive conformation, comprises: determining a distance criterion that, when satisfied, indicates that the atom within the side chain is within a predetermined distance threshold of the another atom within the backbone; and determining that the distance criterion is satisfied for the reactive conformation based on a comparison of the nucleophilic attack distance of the amino acid of the reactive conformation with the predetermined distance threshold.

In some embodiments, the free energy is determined based on analysis of free energy profiles of the dihedral angle for the backbone and the dihedral angle for the side chain of the amino acid in the reactive conformation, and wherein the free energy profiles in spaces of the dihedral angle for the backbone and the dihedral angle for the side chain are calculated from bin populations.

In some embodiments, the predicting the probability of the side chain of the amino acid being trapped in the reactive conformation comprises: determining an energy criterion that, when satisfied, indicates that the free energy of the dihedral angle for the backbone and the dihedral angle for the side chain of the amino acid are within a predetermined energy threshold; and determining that the energy criterion is satisfied for the reactive conformation based on a comparison of the free energy of the dihedral angle for the backbone and the dihedral angle for the side chain of the amino acid in the reactive conformation with the predetermined energy threshold.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims

### BRIEF DESCRIPTION OF THE DRAWINGS

The present disclosure is described in conjunction with the appended figures:

FIG. 1 shows a representation of an exemplary cleavage reaction according to various embodiments;

FIGS. 2A and 2B show two idealized reactive conformations (A and B) and the dihedral angles that minimize the distance for the nucleophilic attack of the asparagine (Asn) side chain nitrogen on backbone carbonyl according to various embodiments;

FIGS. 3A-3F shows free energy profiles along backbone dihedral angles Ψ and two dimensional free energy landscapes along side-chain dihedral angles x₁ and x₂, computed from a 1.5 us molecular-dynamics trajectory according to various embodiments;

FIG. 4 shows a process for generating a probability of a cleavage reaction based on a molecular-dynamic simulation and assessment of molecular spatial properties according to various embodiments;

FIG. 5 shows an example computing device suitable for use with systems and methods for molecular dynamic simulations according to various embodiments;

FIGS. 6A-6C show extraction ion chromatograms for a native peptide bearing the CDR-L3 sequence (FIG. 6A), the N-terminal hydrolysis product (FIG. 6B), and the C-terminal hydrolysis product (FIG. 6C) according to various embodiments;

FIGS. 7A-7D show MS1 spectra for N-terminal hydrolysis product eluting at 98.0 min (FIG. 7A), MS1 spectra for N-terminal hydrolysis product eluting at 98.8 min (FIG. 7B), Theoretical MS1 spectra for Asn N-terminal hydrolysis product (FIG. 7C), and Theoretical MS1 spectra for Asp N-terminal hydrolysis product (FIG. 7D) according to various embodiments;

FIGS. 8A and 8B show Asn-Pro peptide bond hydrolysis in Fab2 according to various embodiments; and

FIG. 9 shows the rate of Asn-Pro peptide hydrolysis in test antibodies according to various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

The present disclosure describes techniques for predicting the likelihood that a peptide bond (e.g., a peptide bond between asparagine (Asn) and bulk residue such as proline (Pro)) for a given polypeptide molecule is susceptible to a cleavage reaction. A given polypeptide may have any number of multiple conformations some of which are reactive conformations and some of which are nonreactive conformations. A cleavage reaction that would result in degradation of the polypeptide can include a reaction between multiple atoms (e.g., the nucleophilic attack of the side chain nitrogen on a backbone carbonyl or a nucleophilic attack of nitrogen of a backbone on the γ-carbon of the side chain). Whether the polypeptide takes on a reactive conformation such that the atoms can react in a cleavage reaction depends on a number of factors including physical proximity of the atoms (e.g., the nucleophilic attack distance (dN)), free energy profiles for dihedral angles of the polypeptide, steric hindrances due to steric bulk, as well as environment conditions such as pH and accessibility of a solvent.

### I.A. Inter Atom Distance Reaction Constraint

Whether a reaction between two atoms of a molecule (e.g., a nucleophilic attack on one of the two atoms) occurs can depend a proximity of the two atoms. In some instances, spatial characteristics of a peptide conformation can include absolute or relative atom positions and/or the distance between two atoms. In some instances, spatial characteristics can include other geometry-associated information that can influence or determine how close two atoms in a molecule are to each other (and thus whether a reaction can occur), such as an angle between multiple atoms or a dihedral angle pertaining to some or all the atoms involved in the reaction (e.g., multiple dihedral angles of the amino-acids neighboring susceptible sites such as susceptible hydrolysis sites). For example, the spatial characteristics can include: (i) a single backbone dihedral angle Ψ defined by 4 atoms of the backbone (composed of Nₙ—C^{α}—C—Nₙ₊₁ atoms), and (ii) two side chain dihedral angles x₁ defined by 4 atoms of the side chain (composed of C—C^{α}—C^{β}—C^{γ} atoms) and x₂ defined by 4 atoms of the side chain (composed of C^{α}—C^{β}—C^{γ}—O atoms), which can be used to estimate a distance between a side chain nitrogen atom and a γ-carbon of the backbone.

The dihedral angles can be estimated by defining a space that corresponds to one dihedral angle (e.g., Ψ) along one axis of the space, another dihedral angle (e.g., x₁) along another axis of the space, and another dihedral angle (e.g., x₂) along another axis of the space. Multiple regions within the space may be defined based on spatial characteristics, with each region being associated with a predicted reaction probability that may include a numerical probability, a categorical probability (e.g., very low, low, moderate, high) or a binary probability. For example, a first region can correspond to particular ranges of the dihedral angles (e.g., Ψ, x₁ and x₂) that would configure the polypeptide such that a distance between two atoms that may participate in a nucleophilic attack is minimized or below a threshold (e.g., 2 ångströms or 3 ångströms). Meanwhile, a second (e.g., remaining) region can correspond to particular ranges of the dihedral angles that would configure the polypeptide such that the two atoms are separated by more than the threshold and thus unlikely to participate in a nucleophilic attack.

### I.B. Steric Hindrance and Free Energy Constraints

The probability of the cleavage reaction can be explored via determining the side chain conformations in molecular dynamic model simulations. If the three-dimensional structure of the polypeptide restricts the side chain in a nonreactive conformation, it would energetically be unfavorable for the side chain to access a reactive conformation. Therefore, the presence of steric hindrances can result in prohibitively high free energy barriers for accessing the reactive conformation. A free energy analysis along side chain dihedral angles can reveal whether the rotation around the dihedral angles toward a reactive conformation is limited by the steric hindrance, and thus the degradation is disabled. An in silico approach can be particularly useful for risk assessment when no experimental data is available. However, even though the ability to identify and access a reactive conformation is important for determining whether a cleavage reaction is likely to occur, it is not absolutely determinative of whether the cleavage reaction will occur. In other words, a side chain might be able to access a reactive conformation, but does not react because the cleavage reaction might not be energetically favorable.

One or more regions (corresponding to a reaction probability) can thus be defined via steric effects and/or energy profile ranges so as to indicate an energy barrier property (e.g., the presence of sufficient steric hindrances that can result in prohibitively high free energy barriers for accessing the reactive conformation) to predict the degradation reaction, in combination with aforementioned structural conformation. It will be appreciated that regions may be separately defined to represent steric hindrance and/or free energy constraints. Thus, a molecular dynamic simulation may be conducted that predicts a likelihood that the polypeptide will transition into a reactive conformation likely to undergo a cleavage reaction. This prediction can include identifying spatial features that make a polypeptide susceptible to particular cleavage reactions and using a molecular dynamics simulation with defined steric hindrance and/or free energy to predict a likelihood that the polypeptide will transition to a conformation that has those spatial features.

### I.C. Solvent Accessibility Reaction Constraint

Even if the inter-atom distance criterion is satisfied (e.g., based on an assessment of dihedral angles of the backbone and side chain) and the free energy criterion is satisfied (e.g., based on an assessment of free energy profiles for dihedral angles of the backbone and side chain), chemical degradation does not occur without a solvent. Thus, an additional chemical-degradation constraint can require that a water molecule (or other solvent) be accessible for hydrolysis. A constraint may be implemented by tracking a quantity of water molecules throughout a simulation. Thus, the simulation may track positions of each of multiple solvent molecules (e.g., and potentially each atom of each of multiple solvent molecules) in addition to tracking positions of individual atoms of the polypeptide. At each time step, it can be determined whether a solvent molecule is within a predefined distance from a particular site on the polypeptide (e.g., a backbone amide site of the polypeptide molecule). Some conformations may inhibit solvent molecules from accessing the particular polypeptide sites as a result of (for example) folds within the polypeptide.

### I.D. Environment Constraint

Therapeutic agents such as mAbs and antibody fragments can be susceptible to chemical and physical instability that can limit their utility. This can be quite concerning if residues of the complementarity determining regions (CDR) are labile since chemical changes at these sites are more likely to have an impact on potency. Development of effective disease treatments using protein therapeutics requires that the therapeutic agent show sufficient stability under both formulation and physiological conditions to be useful. Although advances have been made in in silico testing, more often a thermal stress challenge is applied in vitro to rank candidate molecules suitability for further development. Since mAbs generally have basic isoelectric points, and because deamidation rates increase with pH, antibodies are usually formulated and tested for stability under slightly acidic (pH 5-6) conditions to increase solubility and slow degradation.

This approach can be useful to select candidates with good shelf-life under typical formulation conditions; however, molecules with poor stability under physiological ionic strength and pH (˜7.4) conditions may be missed. Thus, an additional chemical-degradation constraint can require that a certain pH or pH range be present. A constraint may be implemented by tracking the pH of the reaction throughout a simulation. Thus, the simulation may track pH conditions in addition to tracking positions of individual atoms of the polypeptide. At each time step, it can be determined whether the pH is within a predefined range. Some conformations (reactive or otherwise) may more or less predominant as a result of (for example) the current pH of the environment in which the reaction is occurring. It will be appreciated that other types of environmental factors are contemplated to infer other variables affecting conformations. For example, alternatively or additionally, a temperature constraint may be used in combination with other factors such as pH and spatial characteristics to predict a likelihood that the polypeptide will transition into a reactive conformation likely to undergo a cleavage reaction.

### I.F. Simulation and Constraint Usage

By detecting the predicted degradation of a polypeptide, that polypeptide may be passed over in favor another polypeptide with similar therapeutic effect but without such a degradation handicap, or the polypeptide may be coupled with an approach to mitigate the undesired effects of the degradation. One approach for predicting whether a given molecule will degrade is to execute a simulation. However, chemical degradation can involve sub-atomic interactions, covalent-bond formation and covalent-bond breakage. It is not possible to simulate these types of events using conventional molecular dynamics. Some techniques have predicted a reaction probability based on which amino acid motifs are present in a molecule. While reaction probabilities can differ dramatically across motifs, a motif's impact can depend on its location within a molecule (e.g., as to whether the motif is on a heavy chain or light chain and its position within a chain). Even for motifs that are considered highly stable, experimental data identifies some cases in which a reaction occurs at the motif despite the relative general stability.

To address these limitations and problems, the techniques described herein implement molecular-dynamics simulation techniques and molecular-geometry techniques to generate reaction probabilities. One or more iterations of a molecular-dynamics simulation can simulate how a polypeptide's conformation changes in time. A reaction probability can be generated for one or more conformations based on spatial characteristics (e.g., which can determine whether various reaction constraints are satisfied). For example, with respect to each conformation generated by a molecular dynamics simulation, spatial characteristics of the polypeptide in the conformation can be used to determine whether the inter-atom distance reaction constraint and the energy profile constraint are satisfied, which may then indicate the polypeptide having the conformation would be ripe for participation in a cleavage reaction. Solvent and environment inclusive modeling can be used to estimate a proportion of the polypeptides molecules favorably configured for reaction that have access to and react with a solvent molecule. Based on a fraction of the simulation-generated polypeptide conformations for which each constraint is satisfied, an output can be generated that indicate whether, an extent to which and/or a speed at which a given polypeptide chemically degrades to the particular product of interest. Thus, simulation-based techniques disclosed herein can generate predicted reaction susceptibility based on molecular dynamics and analyses of three-dimensional structures of various conformations of a polypeptide (e.g., rather than on conformation-independent data corresponding to identities of amino groups in the polypeptide).

One illustrative embodiment of the present disclosure is directed to a computer-implement method comprising: determining, for a polypeptide conformation of a polypeptide comprising an amino acid having a side chain and a backbone, a dihedral angle for the backbone and a dihedral angle for the side chain of the amino acid while in the polypeptide conformation; determining a nucleophilic attack distance between two atoms, functional groups, or a combination thereof of the amino acid while in the polypeptide conformation based on the dihedral angle for the backbone and the dihedral angle for the side chain, where one of the two atoms or functional groups is in the side chain of the amino acid and another of the two atoms or functional groups is in the backbone of the amino acid; determining, based on the nucleophilic attack distance of the amino acid while in the polypeptide conformation, that the polypeptide conformation is a reactive conformation that is susceptible to a cleavage reaction; in response to determining the polypeptide conformation is the reactive conformation, determining a free energy of the dihedral angle for the backbone and the dihedral angle for the side chain of the amino acid while in the reactive conformation; and predicting a probability of the side chain of the amino acid being trapped in the reactive conformation based on the free energy of the dihedral angle for the backbone and the dihedral angle for the side chain of the amino acid. In some instances, the method further comprises predicting a probability of the polypeptide to chemically degrade as a result of the side chain of the amino acid being trapped in the reactive conformation.

Another illustrative embodiment of the present disclosure is directed to a computer-implement method comprising: generating a representation of a polypeptide comprising an amino acid having a side chain and a backbone; performing a molecular-dynamics simulation using the representation, wherein a result of the performance of the molecular-dynamics simulation includes a set of polypeptide conformations for the polypeptide; determining, for each polypeptide conformation of the set of polypeptide conformations, a spatial characteristic of the amino acid while in the polypeptide conformation, wherein the spatial characteristic includes a dihedral angle for the backbone and a dihedral angle for the side chain; estimating a nucleophilic attack distance between two atoms, functional groups, or a combination thereof of the amino acid of each polypeptide conformation based on a combination of the dihedral angle for the backbone and the dihedral angle for the side chain, where one of the two atoms or functional groups is in the side chain of the amino acid and another of the two atoms or functional groups is in the backbone of the amino acid; identifying, based on the nucleophilic attack distance of the amino acid of each polypeptide conformation, at least one reactive conformation that is susceptible to a cleavage reaction; determining a free energy of the dihedral angle for the backbone and the dihedral angle for the side chain of the amino acid in the at least one reactive conformation; and predicting a probability of the side chain of the amino acid being trapped in the at least one reactive conformation based on the free energy of the dihedral angle for the backbone and the dihedral angle for the side chain of the amino acid. In some instances, the method further comprises predicting a probability of the polypeptide to chemically degrade as a result of the side chain of the amino acid being trapped in the at least one reactive conformation.

### II. Definitions

The term “polypeptide”, as used herein, is used to refer to polymers of amino acids of any length and can include a protein, DNA and/or RNA. The polymer can include a protein including any protein modality, such as an amino acid substituted (un-natural amino acid), alternate glycation, protein, DNA complex and/or virus surface-coat protein. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “chemical degradation”, as used herein, is used to refer to a process by which a molecule (e.g., a polypeptide molecule) is broken down into two or more fragments. In the context of a polymer, chemical degradation can include a full depolymerization of the polymer to corresponding monomers or a partial depolymerization (e.g., to one or more oligomers and potentially one or more other chemical substances). Chemical degradation can include a particular type of chemical process, such as tryptophan oxidation, methionine oxidation, ASN-PRO clipping, asparagine deamidation or aspartate isomerization.

The term “nucleophilic substitution or attack”, as used herein, is a fundamental class of reactions in which an electron rich nucleophile selectively bonds with or attacks the positive or partially positive charge of an atom or a group of atoms (e.g., a functional group) to replace a leaving group. The positive or partially positive atom is referred to as an electrophile.

The term “nucleophilic attack distance”, as used herein, is the average (or mean or median or other similar metric) distance (e.g., angstrom) between the electron rich nucleophile and the atom or a group of atoms (e.g., a functional group) with the positive or partially positive charge.

The term “multiple conformations”, as used herein means a given polypeptide may have any number of spatial arrangement of atoms some of which are reactive conformations and some of which are nonreactive conformations.

The term “reactive conformation”, as used herein, is a conformation of an amino acid or polypeptide wherein the amino acid or polypeptide is prone or susceptible to nucleophilic substitution or attack.

The term “nonreactive conformation”, as used herein, is a conformation of an amino acid or polypeptide wherein the amino acid or polypeptide is not prone or susceptible to nucleophilic substitution or attack, e.g., due to steric hindrance.

As used herein, when an action is “based on” something, this means the action is based at least in part on at least a part of the something.

The terms “substantially,” “approximately” and “about”, as used herein, are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

### III. Exemplary Dependency of Reaction Occurrence

Under physiological conditions asparagine (Asn) residues are susceptible to deamidation, in which the amide side chain is hydrolyzed to form a free carboxylic acid. The rate limiting step for this reaction is formation of a five-membered succinimide ring intermediate. However, it has been shown that peptides containing Asn followed by bulking residues such as proline (Pro) are susceptible to hydrolytic cleavage of the peptide backbone between the two residues. Mass spectral data for the Asn-Pro site identified in the complementarity determining region 3 of the light chain CDR-L3 of at least one mAb resulted in the identification of three peptides related to this site: the native tryptic peptide containing the Asn-Pro site, the N-terminal hydrolysis product peptides containing Asn and iso-Asn, and the C-terminal hydrolysis product peptide. Identification of the N-terminal hydrolysis products containing Asn and iso-Asn rather than Asp and iso-Asp suggest that formation of the succinimide intermediate is the result of an attack of the side chain amide nitrogen on the peptide bond carbonyl. This COOH-terminal succinimide intermediate can then open up to form the observed N-terminal hydrolysis products containing Asn or iso-Asn.

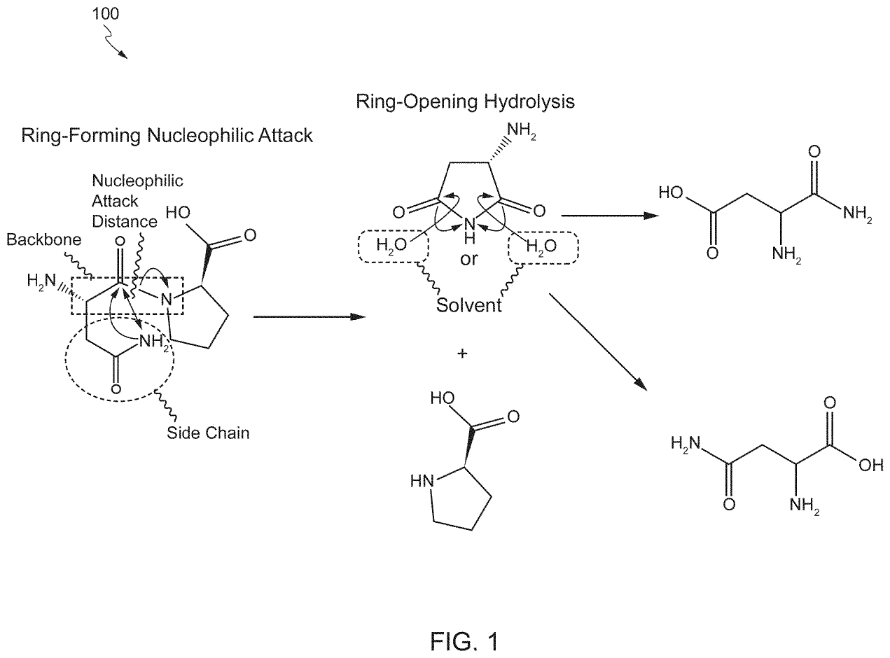

FIG. 1 shows a representation of this exemplary cleavage reaction, which can produce a chemically degraded product. More specifically, FIG. 1 depicts a representation of a Asn residue comprising a side chain with a backbone. If the side chain nitrogen atom and the γ-carbon of the backbone are in sufficiently close proximity and free energy profiles of the dihedral angles of the backbone and side chain are favorable for a transition to a reactive conformation, then the polypeptide molecule is susceptible to a nucleophilic attack of the side chain amide nitrogen on the peptide bond carbonyl. The metastable COOH-terminal succinimide (cyclic imide) intermediate can be produced as a result of the nucleophilic attack. This succinimide intermediate can then open up and if a solvent is accessible to the succinimide intermediate, the succinimide hydrolyzes to a mixture of asparagine and iso-asparagine linkages. With respect to an asparagine residue, the polypeptide may maintain its target characteristics. However, with respect to an iso-asparagine residue, a conformation of the protein and its electrostatic properties can be changed relative to the original polypeptide. If a simulation can reliably predict a probability that a polypeptide will chemically degrade to an undesired product, polypeptides and/or formulations may be selected accordingly to minimize the undesired chemical degradation and maintain an active polypeptide having a target functionality.

### IV. Inter-Atom Distance and Energy Profile Constraint Implementation

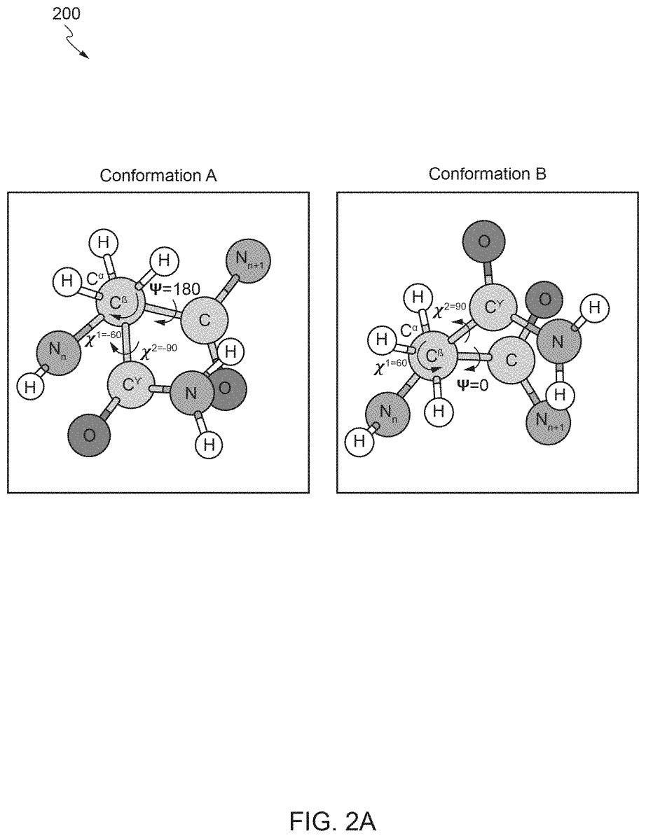

As described herein, the degradation pathway in Asn-Pro hydrolysis proceeds via the nucleophilic attack of the Asn side chain nitrogen on backbone carbonyl. The prerequisite for this process is for the Asn side chain to adopt a reactive conformation that reduces the nucleophilic attack distance (dN) between the Asn side chain nitrogen and the side chain carbonyl. The distance d_{N} is mainly characterized by the combination of the backbone dihedral angle Ψ (composed of Nₙ—C^{α}—C—Nₙ₊₁ atoms), and the side chain dihedral angles 1ₓ_{₁} (composed of C—C^{α}—C^{β}—C^{γ} atoms; note that this is different from conventional chi1, which typical refers to the side-chain dihedral angle composed of N—C^{α}—C^{β}—C^{γ}) and x₂ (composed of C^{α}—C^{β}—C^{γ}—O atoms). As shown in FIGS. 2A and 2B (FIG. 2A shows a ASN-PRO peptide within a protein structure; and FIG. 2B shows a ASN-PRO peptide within a protein structure and the proline ring is visible), for an Asn residue, the distance d_{N} may be minimized in two particular combinations of the dihedral angles: first, when the backbone dihedral angle is extended with Ψ>120 and the side chain dihedral angles are x₁˜−60° and x₂˜−90° (conformation A in FIGS. 2A and 2B), second, when the backbone dihedral angle is in a compact angle with Ψ<−60 and x₁˜60° and x₂˜90° (conformation B in FIGS. 2A and 2B). More specifically, whether a given atom will attack or react with another atom depends on their proximity to one another (nucleophilic attack distance (dN)). In some instances, atoms' positions and angles to one another may be tracked throughout a simulation, and thus, the distance d_{N} can also be tracked. In other instances, dihedral angles can be tracked throughout the simulation, which can be used to infer or estimate whether the atoms are sufficiently close to react. FIGS. 2A and 2B show how three dihedral angles Ψ, x₁ and x₂ affect a distance d_{N} between a side chain nitrogen atom and a γ-carbon of the backbone.

In some instances, a molecular-dynamics simulation may be performed using a representation of a polypeptide having one or more side chains. A result of the performance of the molecular-dynamics simulation may include a set of polypeptide conformations, each polypeptide conformation of the set of polypeptide conformations identifying, for each atom in the polypeptide, a position of the atom. For each polypeptide conformation of the set of polypeptide conformations, one or more spatial characteristics are determined of an amino acid (e.g., Asn) of the polypeptide while in the polypeptide conformation. The one or more spatial characteristics may include multiple inter-atom distances, multiple angles, and/or multiple dihedral angles. In certain instances, the spatial characteristic includes a dihedral angle for the backbone and two dihedral angles for the side chain of the amino acid. A nucleophilic attack distance dN may be calculated or estimated between two atoms, functional groups, or a combination thereof of the amino acid of each polypeptide conformation based on the spatial characteristics (e.g., a combination of the dihedral angle for the backbone and the two dihedral angles for the side chain). Once the nucleophilic attack distance of the amino acid of each polypeptide conformation are calculated or estimated, at least one reactive conformation that is prone or susceptible to nucleophilic substitution or attack may be identified for the polypeptide.

In order to use the dihedral angles and nucleophilic attack distance dN for identifying at least one reactive conformation, a distance criterion may be determined that corresponds to a threshold nucleophilic attack distance across a combination of values for the spatial characteristics (e.g., the dihedral angles Ψ, x₁ and x₂). For example, a predetermined distance threshold (e.g., a minimum nucleophilic attack distance) may be defined between 1.0 Å and 4.0 Å (e.g., 2.5 Å) in order to determine whether the distance criterion is satisfied. The distance criterion may be satisfied when the spatial characteristics (e.g., the dihedral angles Ψ, x₁ and x₂) result in a nucleophilic attack distance dN that is equal to or less than the predetermined distance threshold. When the distance criterion is satisfied, a conformation satisfying the predetermined distance threshold is identified as a reactive conformation. The distance criterion may not be satisfied when the spatial characteristics (e.g., the dihedral angles Ψ, x₁ and x₂) result in a nucleophilic attack distance dN that is greater than the predetermined distance threshold. When the distance criterion is not satisfied, a conformation is identified as a nonreactive conformation. It will be appreciated that satisfaction of the distance criterion for identifying at least one reactive conformation may be determined using alternative techniques, for example, a comparison of the values for the spatial characteristics (e.g., the dihedral angles Ψ, x₁ and x₂) to independent ranges or thresholds indicative of an atom or functional group within the polypeptide backbone chain being within a predefined distance of an atom or functional group of the side chain.

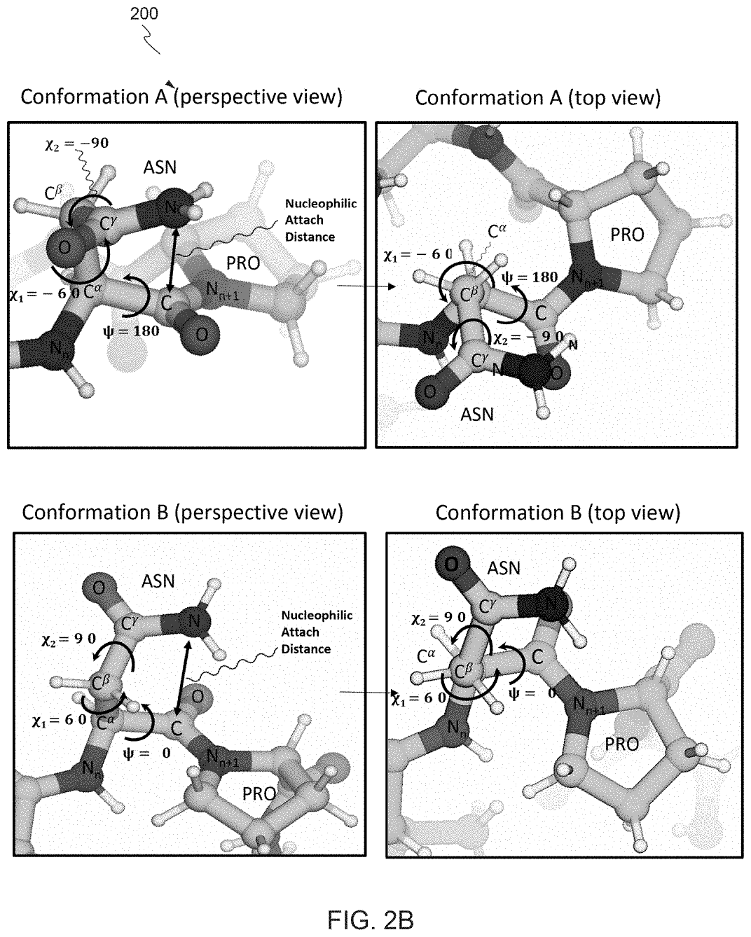

In some instances, the free energy of the side chain along the dihedral angles may be calculated to gain mechanistic insight into the role of side chain conformation on the rate of hydrolysis. The free energy values can be generated via a molecular-dynamics model. FIGS. 3A-3F show the free energies profiles (calculated from bin populations) for backbone dihedral angle (Ψ) and side chain dihedral angles (x₁ and x₂) obtained from molecular-dynamics model simulations. Conformations with low free energy values (represented by darker shades) are more stable than conformations with high free energy values (represented by lighter shades), such that it is more likely that a molecule will be in the conformation. Lower free energies correspond to higher populations (number of frames in the simulation when the confirmation is a reactive conformation), and thus a higher probability of finding the side chain in the given combination of dihedral angles for a reactive conformation. The circles identify particular dihedral-angle ranges that, geometrically, position the nitrogen of a side chain and the γ-carbon of the backbone within a minimized nucleophilic attack distance (e.g., 2 or 3 ångströms). If the circled regions do not include conformations associated with low free energy values, the outputs indicate that the polypeptide is unlikely to chemically degrade as a result of conformations of the polypeptide molecule not bringing the said chain nitrogen atom sufficiently close to the γ-carbon of the back bone to react.

Each of the free energies profiles in FIGS. 3A-3F corresponds to a simulation using a particular polypeptide structure. Notably, the free energies profiles for FIGS. 3A-3D indicate that the corresponding polypeptide (Fab1, Fab2, Mab3, and Mab4) is likely to have conformations in which a distance between the nitrogen of the side chain and the γ-carbon of the backbone is at a minimized nucleophilic attack distance. Meanwhile, the polypeptides (Mab5 and Fab6) corresponding to free energies profiles of FIGS. 3E and 3F is unlikely to be in conformations for which the atoms are in this proximity. More specifically, the free energies profiles along show that Fab2 mainly adopts a compact backbone dihedral angle (Ψ<−60), whereas the other structures Fab1, Fab6, Mab3, Mab4, and Mab5 adopt an extended conformation (Ψ>120). Therefore, the reactive conformation corresponds to conformation B in Fab2 and conformation A in the structures Fab1, Mab3, Mab4, Mab5, and Fab6. The free energies for conformation A are very low for Fab1, Mab3 and Mab4 (0.94, 0.96, and 1.06 kcal/mol, respectively), which is consistent with observed high experimental hydrolysis rates for these structures (13, 15, and 15%/week, respectively). In contrast, the free energy of conformation A is relatively high for Mab5 (1.56 kcal/mol), and is the highest for Fab6 (2.66 kcal/mol), which is in good agreement with observed low experimental rates for these molecules (5 and 0%/week, respectively). However, the agreement with the free energy of reactive conformation and experimental rate is poorer for Fab2, while the free energy of reactive conformation is very small (0.75 kcal/mol), the experimental rate is not as high (9%/week). It is suspected that even though the side chain adopts a reactive conformation, the chemical reaction is not energetically favorable for this case. Thus, in some instances, it may be beneficial to include reaction energy as a constraint within the molecular-dynamics model simulations.

In some instances, the probability of the side chain of an amino acid of a polypeptide being trapped in a reactive conformation is predicted based on the free energy profile of one or more dihedral angles for the backbone and/or the side chain of the amino acid. If the three dimensional structure of the polypeptide restricts the side chain in a nonreactive conformation, it would energetically be unfavorable for the side chain to access a reactive conformation. Therefore, the presence of steric hindrances can result in prohibitively high free energy barriers for accessing the reactive conformation. Alternatively, if the three dimensional structure of the polypeptide restricts the side chain in the given combination of dihedral angles for a reactive conformation, the side chain can get trapped in that reactive conformation. Therefore, a free energy analysis along with a dihedral angle and nucleophilic attack distance analysis can reveal whether the rotation around the dihedral angles toward a reactive conformation is limited by the steric hindrance, and thus the degradation is disabled.

In order to use the free energy analysis along with dihedral angles and nucleophilic attack distance, a free energy criterion may be determined that corresponds to reactive conformations associated with a predetermined distance threshold (e.g., a minimum nucleophilic attack distance) across a combination of values for the spatial characteristics (e.g., the dihedral angles Ψ, x₁ and x₂). For example, a first predetermined energy threshold (minimum free energy value) may be defined between 1.0 kcal/mol and 2.0 kcal/mol (e.g., 1.5 kcal/mol) that corresponds to a first conformation having a backbone dihedral angle extended with Ψ>120° and side chain dihedral angles that are x₁˜−60° and x₂˜−90°. Separately, a second predetermined energy threshold (minimum free energy value) may be defined between 1.0 kcal/mol and 2.5 kcal/mol (e.g., 2.0 Kcal/mol) that corresponds to a second conformation having a backbone dihedral angle compact with Ψ<−60 and side chain dihedral angles that are x₁˜60° and x₂˜90°. Alternatively, a global predetermined energy threshold (minimum free energy value) may be defined between 1.0 kcal/mol and 2.5 kcal/mol (e.g., at 2.0 kcal/mol) that corresponds to all conformations having a dihedral angle for the backbone and the two dihedral angles for the side chain of the amino acid that minimize the nucleophilic attack distance. In some instances, the global predetermined threshold (minimum free energy value) may be defined between 1.0 kcal/mol and 2.5 kcal/mol (e.g., at 2.0 kcal/mol) that corresponds to all conformations having a backbone dihedral angle Ψ between 120° and −60° and side chain dihedral angles x₁ between −60° and +60° and x₂between −90° and +90°. The free energy criterion is satisfied when the spatial characteristics (e.g., the dihedral angles Ψ, x₁ and x₂) have a free energy that is equal to or less than the minimum free energy value (e.g., the global predetermined energy threshold). The free energy criterion is not satisfied when the spatial characteristics (e.g., the dihedral angles Ψ, x₁ and x₂) have a free energy that is greater than the minimum free energy value (e.g., the global predetermined energy threshold). It will be appreciated that satisfaction of the free energy criterion for identifying at least one reactive conformation may be determined using alternative techniques, for example, a comparison of the values for the spatial characteristics (e.g., the dihedral angles Ψ, x₁ and x₂) to independent ranges or thresholds indicative of the free energy of the backbone and/or side chain of the amino acid being within predefined free energy values.

The probability of the side chain of an amino acid of a polypeptide being trapped in a reactive conformation may be defined as a numerical probability, a categorical probability (e.g., very low, low, moderate, high) or a binary probability based on the free energy profile of one or more dihedral angles for the backbone and/or the side chain of the amino acid. For example, if a reactive conformation identified by the distance criterion is associated with low free energy values determined by the free energy criterion, then the output may indicate that the side chain of the amino acid of the polypeptide is likely to be trapped in a reactive conformation (the side chain can access a reactive conformation). Alternatively, if a reactive conformation identified by the distance criterion is associated with high free energy values determined by the free energy criterion, then the output may indicate that the energy barrier is too high and the polypeptide will most likely maintain a nonreactive conformation.

Once the probability of the side chain of an amino acid of a polypeptide being trapped in a reactive conformation is determined, it may be possible to predict the probability of the polypeptide to chemically degrade as a result of the reactive conformation of the polypeptide molecule bringing the side chain nitrogen atom sufficiently close to the γ-carbon of the backbone to react. The probability of the he polypeptide chemically degrading (e.g., undergoing nucleophilic attack and a hydrolysis reaction) may be defined as a numerical probability, a categorical probability (e.g., very low, low, moderate, high) or a binary probability based on the probability of the side chain of an amino acid of a polypeptide being trapped in a reactive conformation. For example, if the side chain of the amino acid of the polypeptide is likely trapped in the reactive conformation, the outputs may indicate that the polypeptide is likely to chemically degrade as a result of the reactive conformation of the polypeptide molecule bringing the side chain nitrogen atom sufficiently close to the γ-carbon of the backbone to react. Alternatively, if the energy barrier is too high and the polypeptide will most likely maintain a nonreactive conformation, then the outputs may indicate that the polypeptide is unlikely to chemically degrade or the degradation is disabled. However, even if the polypeptide may take the reactive conformation, the polypeptide may not degraded unless other factors are present (e.g., a solvent is accessible) in addition to the likelihood of the reactive conformation. Accordingly, in some instances, additional constraints may be included as a factor for predicting conformational behavior and the probability of the polypeptide chemically degrading.

### V. Environment and Accessibility Constraint Implementation

The role of environmental factors such as pH, temperature, and accessibility of a solvent on the conformational behavior and/or the chemical degradation of polypeptides may be investigated by molecular dynamics simulations. An environmental factor such as pH and temperature can be defined as a constant within the simulation. In some instances, pH can be defined by calculating all relevant pKa values for the constituent molecules and assigning the dominant protonation state at a given pH. While a conventional molecular dynamics protocol was used here wherein the protonation states are fixed during the simulation, alternatively methods such as constant pH molecular dynamics that allow for the variation of protonation states in the simulation can be used. Alternatively, quantum mechanics/molecular mechanics (QM/MM) methods may be used to add H3O+ and OH— ions for adjusting the pH value. Even if a nucleophilic attack occurs, a polypeptide is not degraded unless the environment has the right conditions and a solvent molecule is accessible. The molecular dynamics simulation may be further configured to simulate the polypeptide in a solvent (e.g., as an explicit solvent or implicit solvent). A solvent-blocking metric can be defined as a number of frames that the amide group binds with a non-solvent group minus a number of frames that the amide group binds with a solvent molecule (e.g., a water molecule). Thus, negative metrics correspond to greater solvent accessibility as compared to positive metrics. Positive metrics may indicate that a geometry of a polypeptide blocks a solvent molecule from reaching the amide group.

### VI. Process for Predicting Reaction Type for Polypeptide

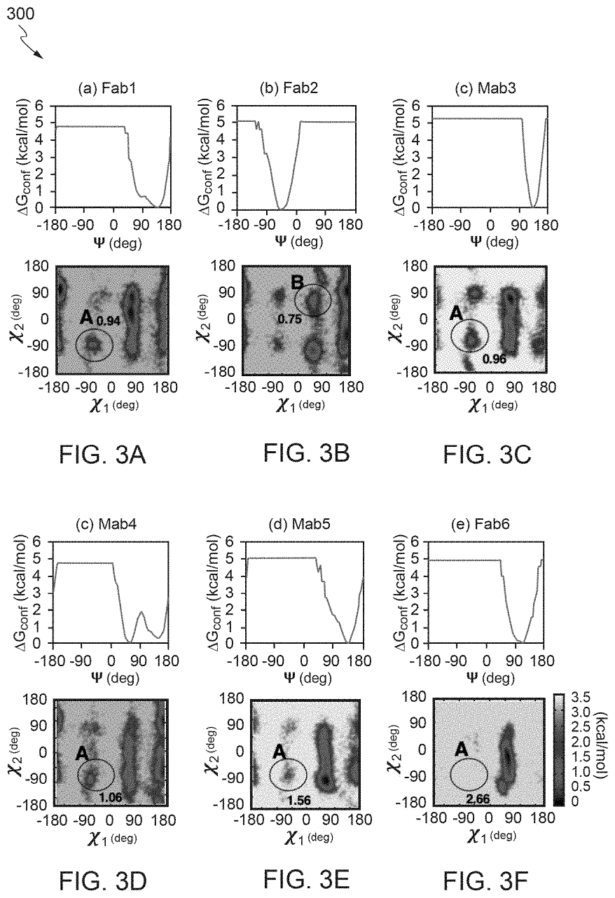

FIG. 4 illustrates a process 400 for generating a probability of a cleavage reaction based on a molecular-dynamic simulation and assessment of molecular spatial properties. Process 400 begins at block 405, where a representation of a polypeptide comprising an amino acid having a side chain and a backbone is generated. The representation can include an identification of atoms, masses, charges and inter-atom connections for a polypeptide (and potentially for a solvent). The representation can further include starting coordinates for each atom of the polypeptide (and potentially for the solvent). The representation may further include constraints to be computationally applied throughout the simulation, such as limits on angles or dihedrals, Van der Wahl terms, free energy, pH, etc.

At block 410, one or more molecular-dynamics simulations are performed using the representation to generate a set of polypeptide conformations. Each polypeptide conformation of the set of polypeptide conformations can correspond to a time step in the simulation(s). Each polypeptide conformation of the set of polypeptide conformations can include, for each atom of the polypeptide, a position of the atom. The set of polypeptide conformations can be determined by calculating forces from particle positions and numerically solving equations of motion. At each time step, in addition to determining a position of each atom, a momenta of each atom can further be estimated.

At block 415, for each polypeptide conformation of the set of polypeptide conformations, determine one or more spatial characteristics of an amino acid while in the polypeptide conformation. The one or more spatial characteristics may include an angle and/or dihedral angle (e.g., Ψ, x₁ and x₂ backbone and/or side chain dihedral angle of an amino acid neighboring a susceptible site). In some instances, the spatial characteristics include a dihedral angle (Ψ) for the backbone and a dihedral angle for the side chain (x₁ or x₂). In other instances, the spatial characteristic includes a dihedral angle (Ψ) for the backbone, a first dihedral angle (x₁) for the side chain, and a second dihedral angle (x₂) for the side chain.

At block 420, a nucleophilic attack distance between two atoms, functional groups, or a combination thereof of the amino acid of each polypeptide conformation is estimated based on one or more spatial characteristics of the amino acid while in the polypeptide conformation. For example, the nucleophilic attack distance between the two atoms, functional groups, or a combination thereof can be estimated using the position of each atom or functional group, the momenta of each atom between atoms or functional groups, and a combination of the dihedral angles for each polypeptide conformation. In some instances, the nucleophilic attack distance between two atoms, functional groups, or a combination thereof of the amino acid of each polypeptide conformation is estimated based on an angle, a dihedral angle, or a combination of the dihedral angles (e.g., the dihedral angle for the backbone and the dihedral angle for the side chain). In some instances, one of the two atoms or functional groups is in the side chain of the amino acid and another of the two atoms or functional groups is in the backbone of the amino acid.

At block 425, at least one reactive conformation that is susceptible to a cleavage reaction is identified based on the nucleophilic attack distance of the amino acid of each polypeptide conformation. In some instances, a distance criterion is determined that may be used to identify the at least one reactive conformation. The distance criterion may correspond to a minimum nucleophilic attack distance across a combination of values for the spatial characteristics (e.g., the dihedral angles Ψ, x1 and x2) that identifies a reactive conformation. For example, a predetermined distance threshold (minimum nucleophilic attack distance) may be defined between 0.0 Å and 3.0 Å (e.g. about 1.5 Å) in order to determine whether the distance criterion is satisfied. Determining whether the distance criterion is satisfied for the at least one reactive conformation may comprise comparing the nucleophilic attack distance of the amino acid of the at least one reactive conformation with the predetermined distance threshold. The distance criterion is satisfied when the nucleophilic attack distance is equal to or less than the predetermined distance threshold. When the distance criterion is satisfied, a conformation satisfying the distance criterion is identified as a reactive conformation. The distance criterion is not satisfied when the nucleophilic attack distance is greater than the predetermined distance threshold. When the distance criterion is not satisfied, a conformation not satisfying the distance criterion is identified as a nonreactive conformation.

At block 430, a free energy is determined of the angle, the dihedral angle, or the combination of the dihedral angles (e.g., the dihedral angle for the backbone and the dihedral angle for the side chain of the amino acid) in the at least one reactive conformation. The free energy may be determined based on analysis of free energy profiles for the angle, the dihedral angle, or the combination of the dihedral angles. In some instances, the free energy profile and landscapes in the space of the angle, the dihedral angle, or the combination of the dihedral angles are calculated from bin populations using

$${G}_{i} = - {k}_{B} T ln ( \frac{Ni}{N max} ) ,$$

where k_{B} is Boltzmann's constant, T is the temperature, Nᵢ is the population of bin i and Nₘₐₓ is the population of the most populated bin. Bins with no population may be given an artificial barrier equivalent to a population of 0.5. At each time step, in addition to determining a position of each atom, the free energy can further be estimated. In certain instances, QM/MM methods may be used to model the free energy.

At block 435, a probability of the side chain of the amino acid being trapped in the at least one reactive conformation may be predicted based on the free energy of the angle, the dihedral angle, or the combination of the dihedral angles (e.g., the dihedral angle for the backbone and the dihedral angle for the side chain of the amino acid) of the amino acid. In some instances, a free energy criterion is determined that that may be used to predict the probability of the side chain of the amino acid being trapped in the at least one reactive conformation. For example, a predetermined energy threshold (minimum free energy value) may be defined between 0.0 kcal/mol and 1.0 kcal/mol that corresponds to all reactive conformations. Determining whether the energy criterion is satisfied for the at least one reactive conformation may comprise comparing the free energy of the angle, the dihedral angle, or the combination of the dihedral angles (e.g., the dihedral angle for the backbone and the dihedral angle for the side chain of the amino acid) in the at least one reactive conformation with the predetermined energy threshold. The free energy criterion is satisfied when the spatial characteristics (e.g., the dihedral angles Ψ, x₁ and x₂) have a free energy that is equal to or less than the predetermined energy threshold. When the free energy criterion is satisfied, the side chain of the amino acid may be predicted to be likely trapped in the reactive conformation (the side chain can access a reactive conformation). The free energy criterion is not satisfied when the spatial characteristics (e.g., the dihedral angles Ψ, x₁ and x₂) have a free energy that is greater than the predetermined energy threshold. When the free energy criterion is not satisfied, the side chain of the amino acid is up against an energy barrier that is too high and the polypeptide may be predicted to likely maintain a nonreactive conformation.

At optional block 440, an environmental and accessibility constraint may be determined that, when satisfied, indicates that an amide group of the polypeptide has above-threshold spatial accessibility to bind with a solvent molecule from a surrounding solvent. The environmental and accessibility constraint is satisfied based on assessing one or more spatial characteristics of the polypeptide in the at least one reactive conformation, one or more environmental factors (e.g., pH or temperature), availability of a solvent molecule, or a combination thereof.

At block 445, a probability of the polypeptide chemically degrading may be predicted as a result of the side chain of the amino acid being trapped in the at least one reactive conformation. For example, if the side chain of the amino acid of the polypeptide is likely trapped in the reactive conformation, the polypeptide may be predicted to likely undergo chemical degradation as a result of the reactive conformation. Alternatively, if the energy barrier is too high and the polypeptide will most likely maintain a nonreactive conformation, then the polypeptide may be predicted to not likely undergo chemical degradation. In certain instances, the probability of the polypeptide chemically degrading may be predicted as a result of the reactive conformation and the environmental and accessibility constraint.

At block 450, the probability of the side chain of the amino acid being trapped in the at least one reactive conformation and/or the probability of the polypeptide chemically degrading is output. For example, the probability of the side chain of the amino acid being trapped in the at least one reactive conformation and/or the probability of the polypeptide chemically degrading may be displayed or transmitted to another device. In some instances, the probability of the side chain of the amino acid being trapped in the at least one reactive conformation and/or the probability of the polypeptide chemically degrading is used to select a polypeptide to be used in a particular manner (e.g., to develop a treatment for a particular condition) and/or to select a particular formulation for the polypeptide (e.g., to restrict water accessing the polypeptide).

In some instances, the probability of the side chain of the amino acid being trapped in the at least one reactive conformation and/or the probability of the polypeptide chemically degrading is used to remove the polypeptide from a list of potential polypeptides to be used as at least part of a therapeutic agent based on the probability of the side chain of the amino acid being trapped in the at least one reactive conformation and/or the probability of the polypeptide chemically degrading.

In some instances, the probability of the side chain of the amino acid being trapped in the at least one reactive conformation and/or the probability of the polypeptide chemically degrading is used to rank the polypeptide lower than another polypeptide in a list of potential polypeptides to be used as at least part of a therapeutic agent based on the probability of the side chain of the amino acid being trapped in the at least one reactive conformation and/or the probability of the polypeptide chemically degrading, where the probability of the side chain of the amino acid being trapped in the at least one reactive conformation and/or the probability of the polypeptide chemically degrading for the another polypeptide is less than the probability of the side chain of the amino acid being trapped in the at least one reactive conformation and/or the probability of the polypeptide chemically degrading for the polypeptide.

### VII. Example Computing Environment

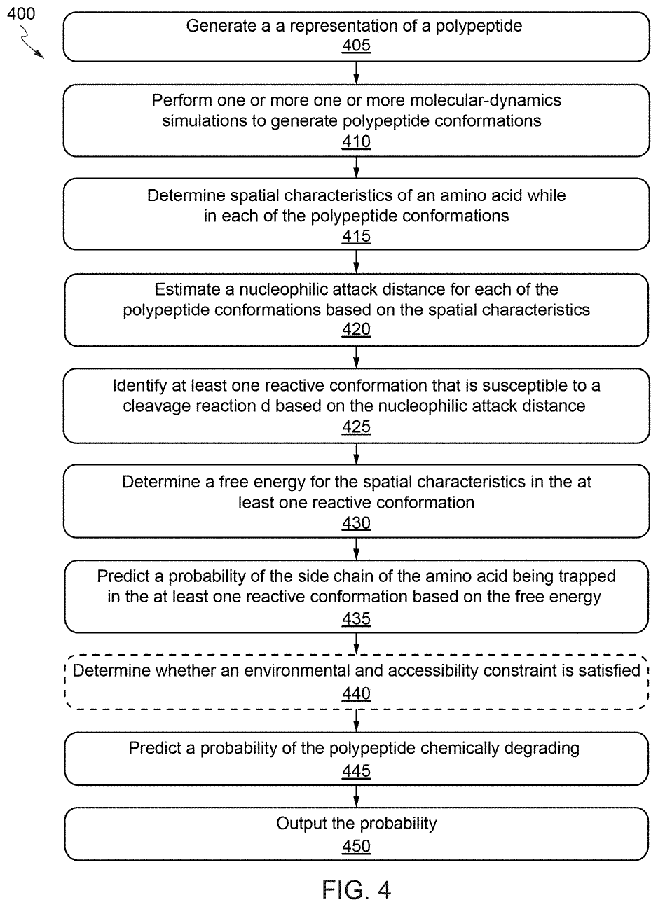

FIG. 5 illustrates an example computing device 500 suitable for use with systems and methods for molecular dynamic simulations according to this disclosure. The example computing device 500 includes a processor 505 which is in communication with the memory 510 and other components of the computing device 500 using one or more communications buses 515. The processor 505 is configured to execute processor-executable instructions stored in the memory 510 to perform one or more methods for molecular dynamic simulations according to different examples, such as part or all of the example method 400 described herein with respect to FIG. 4. In this example, the memory 510 stores processor-executable instructions that provide polypeptide data analysis 520 and predictive analysis 525 for one or more polypeptides of interest, as discussed above with respect to FIGS. 1, 2, 3A-3F, and 4.

The polypeptide data analysis 520 and predictive analysis 525 may be configured to generate polypeptide representations 530 and use those as input in one or more molecular-dynamic simulations to generate reaction probabilities. The molecular-dynamic simulation(s) can be performed using a molecular simulation ensemble 535 that identifies variables of the system that are to be fixed (e.g., a combination of two or more of: number of particles (N), volume (V), energy (E), temperature (T), and pressure (P)). For example, an ensemble can include a microcanonical ensemble (NVE), canonical ensemble (NVT), or a isothermal-isobaric ensemble (NPT). The molecular-dynamic simulation(s) can use an integrator to integrate an equation of motion and a thermostat or barostat to control temperature or pressure throughout the simulation. One or more iterations of the molecular-dynamics simulation can simulate how a polypeptide's conformation changes in time. The simulation(s) may be performed for a particular number of time steps or until a target equilibration is reached.

A reaction probability can be generated for each of multiple conformations based on spatial characteristics (e.g., which can determine whether various reaction constraints are satisfied). For example, with respect to each conformation generated by molecular simulation ensemble 535, spatial characteristics of the polypeptide in the conformation can be used to determine whether the inter-atom distance reaction constraint 540 and the free energy constraint 545 is satisfied, which may then indicate the polypeptide having the conformation would be susceptible to a cleavage reaction. Environment and accessibility constraints 550 can be used to estimate polypeptides molecules favorably configured for reaction that have access to and react with a solvent molecule. Based on a fraction of the simulation-generated polypeptide conformations for which each constraint is satisfied, an output can be generated that indicates whether a side chain of the polypeptide may be trapped in at least one reactive conformation, and/or an extent to which and/or a speed at which a given polypeptide chemically degrades. (It will be appreciated that the simulation may generated multiple outputs of a same conformation or having same spatial properties, which can be uniquely considered.)

The computing device 500, in this example, also includes one or more user input devices 555, such as a keyboard, mouse, touchscreen, microphone, etc., to accept user input. The computing device 500 also includes a display 560 to provide visual output to a user such as a user interface. The computing device 500 also includes a communications interface 565. In some examples, the communications interface 540 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### VIII. EXAMPLES

The systems and methods implemented in various embodiments may be better understood by referring to the following examples.

### VIII.A. Example 1.—Asn-Pro Peptide Bond Hydrolysis in Antibodies: Conformation of CDR-L3 Promotes Reaction

During screening of two antibody Fab fragments as candidates for treatment of ocular disease, fragmentation with concomitant loss of antigen-binding was observed upon incubation at neutral pH and 37° C. Peptide mapping indicated fragmentation occurred at an Asn-Pro site within complementarity determining region three of the light chain (CDR-L3). As has been previously observed in peptides, analysis by mass spectrometry indicates the cleavage is a hydrolysis reaction resulting from attack of the Asn side chain on the peptide carbonyl. A comparison of the rates of CDR-L3 Asn-Pro hydrolysis in five test antibodies showed that, in general, the rate of cleavage is faster than in short, unstructured peptides and suggest the rate is determined by the conformational preference of the segment. In contrast to these results, Asn-Pro found in complementarity determining region two of the heavy chain (CDR-H2), and originating from the germline gene, was not susceptible to this cleavage reaction. Molecular dynamics simulations indicate the Asn residue in susceptible sites populates dihedral angles consistent with attack of the side chain on the peptide carbonyl whereas the Asn at resistant sites does not. From these findings various embodiments discussed herein were derived including techniques for antibody engineering to avoid this instability and an in silico tool for risk assessment of a cleavage reaction.

### VIII.B. Materials and Methods

Size-exclusion chromatography (SEC) was performed using an Agilent 1200 series HPLC system (Santa Clara, Calif.) equipped with a diode array detector (DAD). G6.31 was separated using TSK-GEL G2000SWx1 (7.8×300 mm) column (Tosoh Bioscience, South San Francisco, Calif.). Fab2 samples were diluted to approximately 0.5 mg/mL in mobile phase (0.2 M potassium phosphate, 0.25M potassium chloride pH 6.2). Seventy μL of the sample was injected on to the column and eluted at 25° C. in isocratic mode at a flow rate of 0.5 mL/min for 30 minutes and UV absorption at 280 nm was used for detection. The SEC peaks were divided into monomer, high molecular weight species (HMWS), and fragments. The percent peak area was calculated by dividing the peak area of each group at each time point to the total peak area.

Ion-exchange chromatography (IEC) was performed using an Agilent 1200 series HPLC system on Dionex Propac WCX-10 column (4×250) (Tosoh Bioscience, South San Francisco, Calif.). Mobile phase A (20 mM MES at pH 5.7) and B (200 mM sodium chloride in mobile phase A) were used for separation. A linear gradient starting from 92% solvent A to 34% solvent A at 85 minutes followed by a gradient from 34% solvent A to 0% solvent A at 95 minutes was employed to separate Fab2 charge variant in a total of ˜100 min run time. 75 μL of the sample was injected on to the column and eluted at 25° C. in isocratic mode at a flow rate of 0.8 mL/min and UV absorption at 280 nm was used for detection. The IEC peaks were divided into main peak, acidic peak, and basic peak. The percent peak area was calculated by diving each peak area to the total peak area.

Antigen binding capacity of Fab2 was measured using surface plasmon resonance (SPR) on a Biacore T200 instrument (GE Healthcare, Pittsburgh, Pa.) by using a protocol similar to that described (Tesar et al., 2017, mAbs). Briefly, the antigen was immobilized directly on to the carboxyl methylated dextran sensor chip (CM5) in the range 2000-3000 response units (RU) using amine coupling kit (GE Healthcare, Pittsburgh, Pa.). The binding of antibody Fab fragment to antigen was determined by monitoring the change in the RU before and after injection for 180 s. The sensor chip was regenerated with 10 mM glycine-HCl buffer at pH 2.1 and 30 μL/min flow rate for 30 s. All binding assays were performed at ambient temperature in HEPES buffer (0.01 M HEPES, 0.15 M NaCl, 0.005% (v/v) surfactant P20, pH 7.4]. The antigen-binding concentration was calculated from a standard calibration curve (0.158-5 μg/mL) using a four-parameter fit. The antigen-binding capacity at each time point was normalized to the antigen-binding capacity at t0.

### IV.C. Molecular Dynamics Simulation Details

Modeling builder software (e.g., a modified version of MODELLER) was used to construct the Fab structures from the sequence. The Fab structures were energy minimized to remove steric clashes. The relaxed structures then were analyzed to determine the protonation states for ionizable residues at pH=7.4. Fab structures then were solvated in a octahedron solvent box of TIP3P water with at least 10 Å distance to the edge of the box with periodic boundary conditions. The solute structure was parameterized with a FF14SB force field. The system charge was neutralized with Na+ and Cl− counter ions. Hydrogen Mass Repartitioning was performed on the solute atoms to enable a simulation time step of 4 fs.

An exemplary simulation protocol included the following steps. First, the structures were relaxed with 2000 steps of conjugate-gradient energy minimization, using harmonic restraining potential with the force constant of 10 (kcal/mol/Å²) to restrain the solute to the initial structure. Then the pressure was maintained at 1 atm and the thermostat temperature increased to 300K over the course of 200 ps, while Harmonic positional restraints of strength 10 (kcal/mol/Å²) was applied to the protein structure. The system was then equilibrated for 500 ps with a restraint force constant of 1 (kcal/mol/Å²). All restraints were removed for the production stage. The simulation time step was 4 fs. A cutoff radius of 9 Å was used for range-limited interactions, with particle mesh electrostatics for long-range interactions. The production simulation was carried out using NPT conditions. Langevin dynamics was used to maintain the temperature at 300K with a collision frequency of γ=1 ps⁻¹. The production stage of the molecular-dynamics simulation was performed for 500 ns. During dynamics a SHAKE algorithm was used to constrain all bonds involving hydrogen atoms. For the analyses presented below, snapshots from the molecular-dynamics trajectory were saved every 10 ps.

Default values were used for all other simulation parameters. The GPU implementation of Amber 2015 molecular-dynamics simulation software package with the SPFP precision model was used for exemplary molecular-dynamics simulation. The molecular-dynamics simulation protocol described above was repeated to run 3 independent 500 ns molecular-dynamics simulation simulations per molecule. The trajectories from three simulations were then combined (adding up to 1.5 μs) to be used for the analysis. CPPTRAJ software in AmberTools was used to analyze the trajectories. The free energy profile and landscapes in the space of dihedral angles were calculated from bin populations using

$${G}_{i} = - {k}_{B} T ln ( \frac{Ni}{N max} ) ,$$

where k_{B} is Boltzmann's constant, T is the temperature, Nᵢ is the population of bin i and Nₘₐₓ is the population of the most populated bin. Bins with no population were given an artificial barrier equivalent to a population of 0.5.

### VIII.D. Results

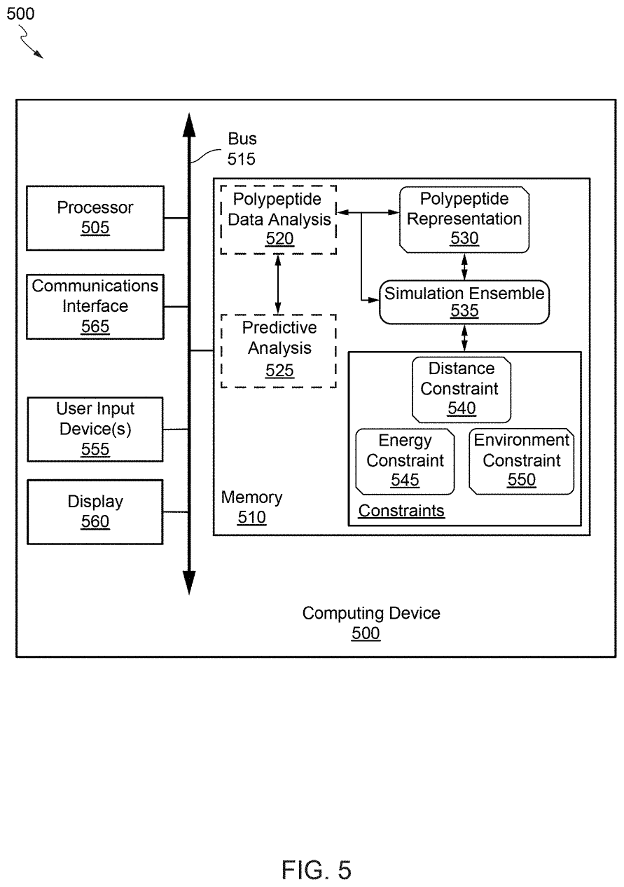

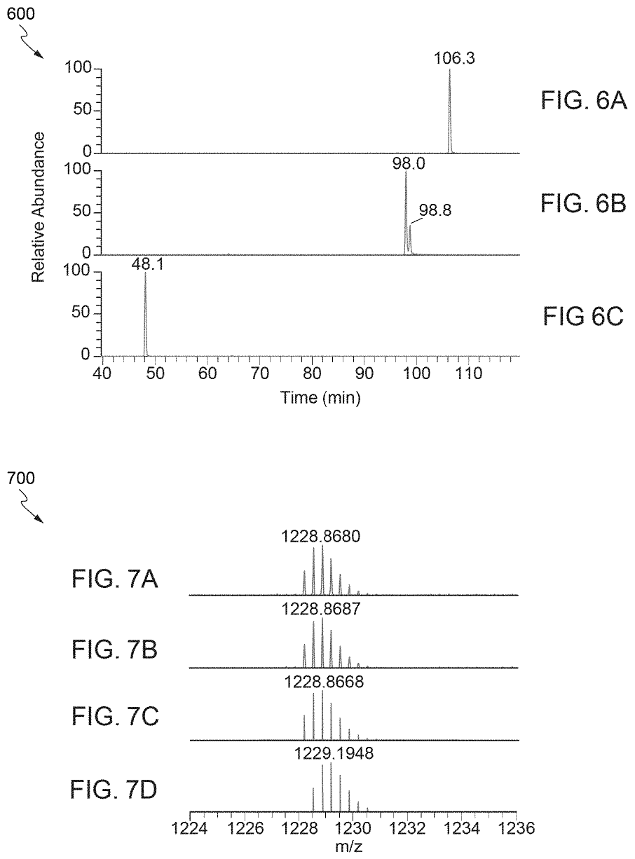

Purified Fab1 was stressed in PBS at 37° C. for 4 weeks. The control and stress samples were then subjected to tryptic digestion followed by reversed phase chromatography separation and mass spectrometric analysis to identify potential degradation products. A labile Asn-Pro site prone to hydrolysis was identified in CDR-L3 of Fab1 (see, e.g., FIGS. 6A-6C). Extracted ion chromatograms (XICs) are shown for the native tryptic peptide containing the Asn-Pro site (FIG. 6A), the N-terminal hydrolysis product peptide (FIG. 6B) and the C-terminal hydrolysis product peptide (FIG. 6C). Two N-terminal hydrolysis products were observed (FIG. 6B) corresponding to Asn and Iso-Asn at the C-terminus of the peptide rather than Asp and Iso-Asp at the C-terminus. The observation of Asn at the C-terminus of the N-terminal hydrolysis product peptide can be seen in FIGS. 7A and 7B, which show the mass spectra corresponding to the two N-terminal hydrolysis products eluting at 98.0 min and 98.8 min, respectively. The theoretical mass spectra for the N-terminal hydrolysis product corresponding to an Asn or Asp at the C-terminus of the peptide are shown in FIG. 7C and 7D, respectively. Tandem mass spectra of the N-terminal hydrolysis product confirms the Asn at the C-terminus of the peptide.

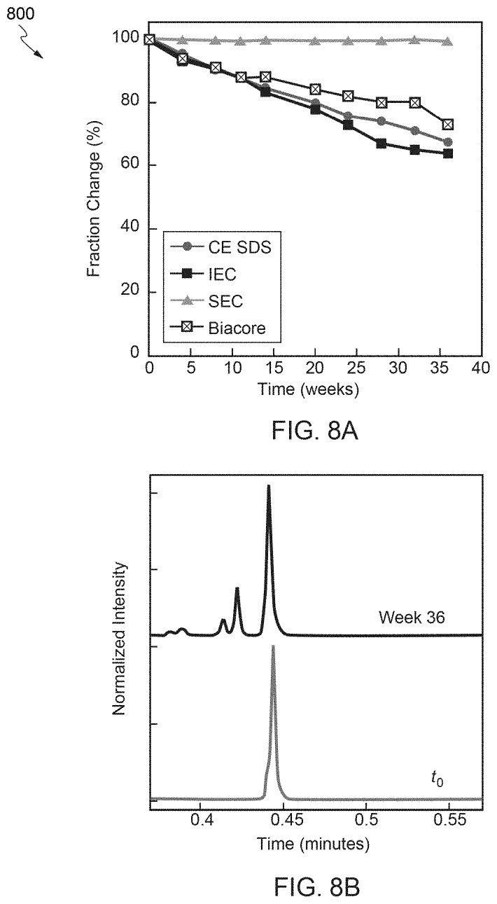

The long-term stability of Fab2 was assessed in PBS (pH 7.4) at 37° C. FIG. 8A summarizes the change in the Fab stability over a 36-week period. Measurement of aggregation using SEC shows that the Fab remains monomeric during the entire stress period. However, measurement of side chain chemical degradation and main chain fragmentation using IEC and CE-SDS respectively suggests that the Fab undergoes slow and steady degradation. The decrease in main peak fraction after 36 weeks as measured by IEC and CE-SDS is 32.7% and 36% respectively. Consequently, the antigen binding capacity measured using SPR decreases by 27% during the same time period. Ion-exchange chromatogram shows that the decrease in IEC main peak is due to an increase in acidic charge variants, presumably due to deamidation reaction. Gel electrophoresis performed under denaturing conditions clearly shows that the decrease in CE-SDS main peak is due to main chain fragmentation (FIG. 8B). Mass spectrometry analysis of the stressed sample confirmed that fragmentation is due to Asn-Pro hydrolysis. However, SEC data shows that under non-denaturing conditions the Fab remains intact despite fragmentation due to Asn-Pro hydrolysis. Addition of a broad spectrum protease inhibitor cocktail (cOmplete™, Roche) to the incubation mixture did not affect the rate of Fab2 light chain fragmentation consistent with hydrolysis resulting from an autolysis event rather than from presence of a trace proteolytic enzyme contamination. Cleavage of an unstructured 17-mer peptide containing an Asn-Pro sequence spiked into neutral pH formulations of Fab2, and incubated at 37° C., was not detected. This is also inconsistent with a protease catalyzed fragmentation.

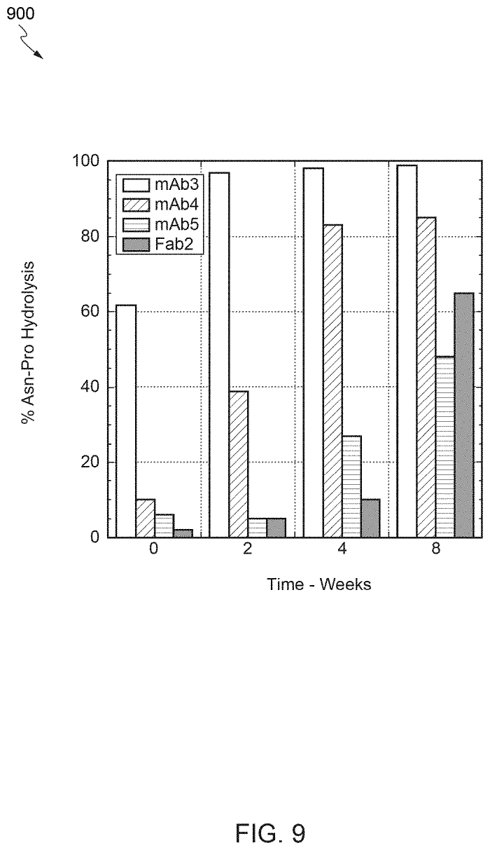

A sequence comparison of a collection of antibodies identified several potential therapeutic candidates with Asn-Pro motif in CDR-L3. In all of these antibodies the Asn-Pro is fixed at position 6-7 but the remainder of the positions in CDR-L3 are varied. Four of these, one antibody Fab fragment and three full length antibodies were chosen for analysis of the rate of Asn-Pro hydrolysis upon thermal stress (37° C.) of protein solutions formulated in PBS. All showed susceptibility of the Asn-Pro peptide bond to hydrolysis with the kinetics of cleavage shown in FIG. 9. Mab3 and Mab4 showed the highest rate of hydrolysis (Table 1), greater than observed for Fab1, whereas the rate of hydrolysis was slower for Fab2, and Mab5 had the slowest rate. Mab3 contained a significant amount of hydrolyzed Asn-Pro in the starting material presumably because the antibody had been previously stored in a neutral pH buffer. Although this data set is too small to delineate adjacent sequence effects on hydrolysis rate, it is noteworthy that sequence variation in CDR-L3 leads to a 3-fold range in hydrolysis. In addition, Fab4, having the CDR sequences of Fab2 grafted into an alternative, non-human framework, showed a 4-fold lower rate (2%/week) of Asn-Pro hydrolysis compared to Fab2 (8%/week). In contrast to these results, analysis of a panel of antibodies having Asn-Pro in CDR-H2 indicated this position was not susceptible to hydrolysis. For example, hydrolysis was not detected for Asn-Pro in CDR-H2 of Fab3 (Table 1) upon thermal stress (40° C.) of protein formulated in PBS.

TABLE 1
CDR-L3 sequences and Asn-Pro hydrolysis
rates at pH 7.4 of training set
antibodies

| | | Hydrolysis |
|---|---|---|
| | CDR-L3 | rate |
| Protein | Sequence | (%/week) |
| Fab1 | QQWSSNPWT | 13 |
| | (SEQ ID NO: 1) | |
| Fab2 | QQGYGNPFT | 8 |
| | (SEQ ID NO: 2) | |
| Mab3 | QQWSSNPYT | 15 |
| | (SEQ ID NO: 3) | |
| Mab4 | QQGINNPLT | 15 |
| | (SEQ ID NO: 4) | |
| Mab5 | QQWSFNPPT | 5 |
| | (SEQ ID NO: 5) | |
| Fab3 | CDR-H2 | ~0 |
| | EINPTSGGTN | (hydrolysis |
| | FNEKFKS | not |
| | (SEQ ID NO: 6) | detected) |
| Fab4 | QQGYGNPFT | 2 |
| [Fab2 | (SEQ ID NO: 7) | |
| CDRs in | | |
| non-human | | |
| framework] | | |

To examine the effect of antibody structure on the rate of Asn-Pro hydrolysis, thermal stress was performed on unstructured, linear peptides representing the CDR-L3 sequence of Fab1 and Mab3. Since it had been previously shown that the rate of hydrolysis in unstructured peptides incubated at 37° C. was slow, accelerated temperature conditions (90 and 70° C.) were performed and the temperature dependence extrapolated to calculate the rate at 37° C. Very slow rates of peptide hydrolysis (Table 2), with half-lives calculated for 37° C. of greater than 2000 days, were observed. As expected, the rate of hydrolysis was slower, by about 3-fold, for incubations performed in pH 5 buffer.

TABLE 2
Rate of Asn-Pro hydrolysis in peptide
mimics of selected antibody CDR-L3
segments at pH 7.4, 90° C.

| | | % Pro |
|---|---|---|
| | | peak |
| | | formation/ |
| Peptide | | day |
| Name | Sequence | at 90° C. |
| Mab3 | QQWSSNPYTFGQ | 2.9 |
| | (SEQ ID NO: 3) | |
| Fab1 | QQWSSNPWTFGQ | 1.0 |
| | (SEQ ID NO: 1) | |
| Fab2 | QQGYGNPFTFGQ | 2.9 |
| | (SEQ ID NO: 2) | |
| Mab4 | QQGINNPLTFGQ | 1.9 |
| | (SEQ ID NO: 4) | |
| Mab5 | QQWSFNPPTFGQ | ND |
| | (SEQ ID NO: 5) | |

ND = not detected

To further investigate the effect of protein structure on Asn-Pro hydrolysis rate, molecular dynamics simulations of the Fab structures were performed. As discussed herein, the degradation pathway in Asn-Pro hydrolysis proceeds via the nucleophilic attack of the Asn side chain nitrogen on backbone carbonyl. The prerequisite for this process is for the ASN side chain to adopt a conformation that minimizes the nucleophilic attack distance dN between the Asn side chain nitrogen and the side chain conbonyl (FIGS. 2A and 2B). The distance d_{N} is minimized in two particular combinations of the dihedral angles: first, when the backbone dihedral angle is extended with Ψ>120 and the side-chain dihedral angles are x₁˜−60° and x₂˜−90° (i.e. conformation A in FIGS. 2A and 2B), second, when the backbone dihendral angle is in a compact angle with Ψ<−60 and x₁˜60° and x₂˜90° (i.e. conformation B in FIGS. 2A and 2B). The free energy of side-chain along the dihedral angles was calculated to gain mechanistic insight into the role of side-chain conformation on the rate of hydrolysis.

FIGS. 3A-3F show the free energies profiles (calculated from bin populations, see Molecular Dynamics Simulation Details section) for backbone dihedral angle (Ψ) and side chain dihedral angles (x₁ and x₂) obtained from 1.5 microsecond MD simulations. Lower free energies correspond to higher probability of finding the side-chain in the given combination of dihedral angles. The free energies profiles along Ψ show that Fab2 mainly adopts a compact backbone dihedral angle (Ψ<−60), whereas the other structures adopt an extended conformation (Ψ>120). Therefore, the reactive conformation corresponds to conformation B in Fab2 and conformation A in other molecules. as indicated by circles in FIGS. 3A-3F. The free energies for conformation A are very low for Fab1, Mab3 and Mab4 (0.94, 0.96, and 1.06 kcal/mol, respectively), which is consistent with the high experimental hydrolysis rates for these structures (13, 15, and 15%/week, respectively). In contrast, the free energy of conformation A is relatively high for Mab5 (1.56 kcal/mol), and is the highest for Fab6 (2.66 kcal/mol), which is in good agreement with low experimental rates for these molecules (5 and 0%/week, respectively). However, the agreement with the free energy of reactive conformation and experimental rate is poorer for Fab2, while the free energy of reactive conformation is very small (0.75 kcal/mol), the experimental rate is not as high (9%/week). It is suspected that, even though the side-chain adopts a reactive conformation, the chemical reaction is not energetically favorable for this case.

### VIII.E. Discussion

Under physiological conditions Asn residues are susceptible to deamidation, in which the amide side chain is hydrolyzed to form a free carboxylic acid. The rate limiting step for this reaction is formation of a five-membered succinimide ring intermediate. However, it has been shown that peptides containing Asn followed by bulking residues such as proline are susceptible to hydrolytic cleavage of the peptide backbone between the two residues. Herein is provided evidence of Asn-Pro motifs susceptible to hydrolytic cleavage of the peptide backbone. Mass spectral data for the Asn-Pro site identified in CDR-L3 resulted in the identification of three peptides related to this site: the native tryptic peptide containing the Asn-Pro site, the N-terminal hydrolysis product peptides containing Asn and iso-Asn, and the C-terminal hydrolysis product peptide. Identification of the N-terminal hydrolysis products containing Asn and iso-Asn rather than Asp and iso-Asp suggest that formation of the succinimide intermediate is the result of an attack of the b-side-chain amide nitrogen on the peptide bond carbonyl. This COOH-terminal succinimide intermediate can then open up to form the observed N-terminal hydrolysis products containing Asn or iso-Asn.

For murine antibodies, or antibodies based on humanization of a murine antibody, Asn-Pro at light chain position 94-95 (Kabat numbering) can arise from selection of a IGKV4 mouse germline gene since some members of this family have Asn-Pro encoded in the un-recombined gene. In contrast, there are no germline encoded 94Asn-Pro95 in human light chain genes such that for human antibodies this motif would come from the V-J joining process of recombination or via somatic hyper mutation. Similarly, Asn-Pro at heavy chain position 52-52a (CDR-2) is found in both human and mouse germline genes of the IGHV1 family. As a consequence, in collections of antibodies 52Asn-Pro52a in CDR-H2 is found more frequently than 94Asn-Pro95 in CDR-L3. The motif in CDR-H2 tends to be less solvent accessible, and more involved in maintaining CDR conformation, as compared to the more solvent accessible CDR-L3 location where the Asn-Pro is more likely to play a direct role in antigen binding. This renders Asn-Pro in CDR-L3 as more susceptible to hydrolysis with cleavage more likely to have an impact on target binding.

Recently, Jain et al. (PNAS, 2017) published a report on biophysical properties of clinical-stage antibodies aimed at setting metrics for “developability”. In their collection of 137 antibodies there are 8 antibodies with 94Asn-Pro95 in CDR-L3. These are muromonab, mAb5, otlertuzumab, rituximab, teplizumab, tovetumab, veltuzumab, and visilizumab. All have nine residue CDR-L3 sequences except for tovetumab that has P95a insertion for a 10 residue CDR. Teplizumab appears to be a humanized version of muromonab whereas veltuzumab has features consistent with a humanized version of rituximab. Notably, teplizumab and visilizumab also have an Asn-Pro in CDR-H2. A subsequent publication (Lu et al., 2018) tested deamidation and isomerization liability for 131 of the antibodies described by Jain et al. and included a pH 8.5 stress test for the 8 antibodies with 94Asn-Pro95 in CDR-L3. Evidence for cleavage of these antibodies at this site was not reported. Using the in silico techniques described herein, it was possible to predict (Table 3) that the light chains of antibodies tovetumab and visilizumab would have cleavage-susceptible bonds whereas the others should be less susceptible to cleavage.

In addition to the data shown for Mab5 in Table 1 and FIG. 9, neutral pH, 37 C stability data was generated for all except muromonab, teplizumab, tovetumab, and visilizumab. Antibodies formulated in PBS, pH 7.4, were incubated for up to 4 weeks at 37° C. and evidence for fragmentation was obtained by mass spectroscopy and capillary electrophoresis sodium dodecyl sulfate (CE-SDS) for both intact and reduced samples. Although rates were not determined, hydrolysis at Asn-Pro in CDR-L3 was detected for veltuzumab but not rituximab or otlertuzumab (Table 3). Stability of the bond in rituximab and otlertuzumab was expected but instability in veltuzumab was not. These results support the conclusion that antibody framework can influence the rate of hydrolysis since veltuzumab and rituximab have the same amino acid sequence in CDR-L3.

TABLE 3
Commercial antibodies with
Asn-Pro in CDR-L3

| | | Light chain |
|---|---|---|
| | CDR-L3 | fragment |
| Protein | Sequence | observed? |
| muromonab | QQWSSNPFT | NT |
| | (SEQ ID NO: 8) | |
| otlertuzumab | QHHSDNPWT | No |
| | (SEQ ID NO: 9) | |
| rituximab | QQWTSNPPT | No |
| | (SEQ ID NO: 10) | |
| veltuzumab | QQWTSNPPT | Yes |
| | (SEQ ID NO: 11) | |
| teplizumab | QQWSSNPFT | NT |
| | (SEQ ID NO: 12) | |
| tovetumab | QQTYSNPPIT | NT |
| | (SEQ ID NO: 13) | |
| visilizumab | QQWSSNPPT | NT |
| | (SEQ ID NO: 14) | |

NT = not tested

Accordingly, it has been demonstrated that hydrolysis of the Asn-Pro peptide bond upon extended thermal stress at pH 7.4 results in loss of antigen binding for Fab2. Since many therapeutic antibodies and antibody fragments are formulated under slightly acidic conditions, where the kinetics of the Asn-Pro cleavage are slower, this reaction may have been underappreciated from studies to determine the shelf-life of antibody formulations. While a lower pH liquid or lyophilized formulation could be used to stabilize against Asn-Pro cleavage in a susceptible antibody, this will not eliminate potential degradation under physiological conditions of neutral pH. In some instances it may be desirable to re-engineer the antibody by amino acid substitution of the labile Asn residue. Indeed, clinical candidates were generated from Fab 1 and Fab2 via Asn-94 substitution that were able to influence the degradation rate. Thus, if the Asn residue is intimately involved in antigen binding, such that the residue is immutable, then changing the sequence context could stabilize the molecule with retention of target-binding affinity. This was demonstrated with the Fab4 molecule, having the CDRs grafted into a non-human framework, that had a 4-fold slower rate of hydrolysis of the labile bond than in the parental antibody Fab2. Alternatively, candidates could be selected for development that lack this sequence motif or, for molecules having Asn-Pro, subjected to in silico risk assessment for this cleavage reaction.

### IX. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
determining, for a polypeptide conformation of a polypeptide comprising an amino acid having a side chain and a backbone, a dihedral angle for the backbone and a dihedral angle for the side chain of the amino acid while in the polypeptide conformation;
determining a nucleophilic attack distance between two atoms, functional groups, or a combination thereof of the amino acid while in the polypeptide conformation based on the dihedral angle for the backbone and the dihedral angle for the side chain, wherein one of the two atoms or functional groups is in the side chain of the amino acid and another of the two atoms or functional groups is in the backbone of the amino acid;
determining, based on the nucleophilic attack distance of the amino acid while in the polypeptide conformation, that the polypeptide conformation is a reactive conformation that is susceptible to a cleavage reaction;
in response to determining the polypeptide conformation is the reactive conformation, determining a free energy of the dihedral angle for the backbone and the dihedral angle for the side chain of the amino acid while in the reactive conformation; and
predicting a probability of the side chain of the amino acid being trapped in the reactive conformation based on the free energy of the dihedral angle for the backbone and the dihedral angle for the side chain of the amino acid.

2. The computer-implemented method of claim 1, further comprising:
generating a representation of the polypeptide; and
performing a molecular-dynamics simulation using the representation, wherein a result of the performance of the molecular-dynamics simulation comprises a set of polypeptide conformations for the polypeptide including the polypeptide conformation.

3. The computer-implemented method of claim 1, further comprising predicting a probability of the polypeptide to chemically degrade as a result of the side chain of the amino acid being trapped in the reactive conformation.

4. The computer-implemented method of claim 3, further comprising outputting the probability of the side chain of the amino acid being trapped in the at least one reactive conformation and/or the probability of the polypeptide chemically degrading.

5. The computer-implemented method of claim 4, further comprising removing the polypeptide from a list of potential polypeptides to be used as at least part of a therapeutic agent based on the probability of the side chain of the amino acid being trapped in the at least one reactive conformation and/or the probability of the polypeptide chemically degrading.

6. The computer-implemented method of claim 4, further comprising ranking the polypeptide lower than another polypeptide in a list of potential polypeptides to be used as at least part of a therapeutic agent based on the probability of the side chain of the amino acid being trapped in the at least one reactive conformation and/or the probability of the polypeptide chemically degrading, wherein the probability of the side chain of the amino acid being trapped in the at least one reactive conformation and/or the probability of the polypeptide chemically degrading for the another polypeptide is less than the probability of the side chain of the amino acid being trapped in the at least one reactive conformation and/or the probability of the polypeptide chemically degrading for the polypeptide.

7. The computer-implemented method of claim 3, wherein the predicting the probability of the polypeptide to chemically degrade includes:
identifying an accessibility constraint that, when satisfied, indicates that an amide group of the polypeptide has above-threshold spatial accessibility to bind with a solvent molecule from a surrounding solvent; and
determining, for the reactive conformation, that the accessibility constraint is satisfied based on assessing one or more spatial characteristics of the polypeptide.

8. The computer-implemented method of claim 1, wherein the determining that the polypeptide conformation is the reactive conformation, comprises:
determining a distance criterion that, when satisfied, indicates that the atom within the side chain is within a predetermined distance threshold of the another atom within the backbone; and
determining that the distance criterion is satisfied for the reactive conformation based on a comparison of the nucleophilic attack distance of the amino acid of the reactive conformation with the predetermined distance threshold.

9. The computer-implemented method of claim 1, wherein the free energy is determined based on analysis of free energy profiles of the dihedral angle for the backbone and the dihedral angle for the side chain of the amino acid in the reactive conformation, and wherein the free energy profiles in spaces of the dihedral angle for the backbone and the dihedral angle for the side chain are calculated from bin populations.

10. The computer-implemented method of claim 1, wherein the predicting the probability of the side chain of the amino acid being trapped in the reactive conformation comprises:
determining an energy criterion that, when satisfied, indicates that the free energy of the dihedral angle for the backbone and the dihedral angle for the side chain of the amino acid are within a predetermined energy threshold; and
determining that the energy criterion is satisfied for the reactive conformation based on a comparison of the free energy of the dihedral angle for the backbone and the dihedral angle for the side chain of the amino acid in the reactive conformation with the predetermined energy threshold.

11. A system comprising:
one or more data processors; and
a non-transitory computer readable storage medium containing instructions which, when executed on the one or more data processors, cause the one or more data processors to perform actions including:
determining, for a polypeptide conformation of a polypeptide comprising an amino acid having a side chain and a backbone, a dihedral angle for the backbone and a dihedral angle for the side chain of the amino acid while in the polypeptide conformation;
determining a nucleophilic attack distance between two atoms, functional groups, or a combination thereof of the amino acid while in the polypeptide conformation based on the dihedral angle for the backbone and the dihedral angle for the side chain, wherein one of the two atoms or functional groups is in the side chain of the amino acid and another of the two atoms or functional groups is in the backbone of the amino acid;
determining, based on the nucleophilic attack distance of the amino acid while in the polypeptide conformation, that the polypeptide conformation is a reactive conformation that is susceptible to a cleavage reaction;
in response to determining the polypeptide conformation is the reactive conformation, determining a free energy of the dihedral angle for the backbone and the dihedral angle for the side chain of the amino acid while in the reactive conformation; and
predicting a probability of the side chain of the amino acid being trapped in the reactive conformation based on the free energy of the dihedral angle for the backbone and the dihedral angle for the side chain of the amino acid.

12. The system of claim 11, wherein the actions further comprise:
generating a representation of the polypeptide; and
performing a molecular-dynamics simulation using the representation, wherein a result of the performance of the molecular-dynamics simulation comprises a set of polypeptide conformations for the polypeptide including the polypeptide conformation

13. The system of claim 11, wherein the actions further comprise predicting a probability of the polypeptide to chemically degrade as a result of the side chain of the amino acid being trapped in the reactive conformation.

14. The system of claim 13, wherein the actions further comprise outputting the probability of the side chain of the amino acid being trapped in the at least one reactive conformation and/or the probability of the polypeptide chemically degrading.

15. The system of claim 14, wherein the actions further comprise removing the polypeptide from a list of potential polypeptides to be used as at least part of a therapeutic agent based on the probability of the side chain of the amino acid being trapped in the at least one reactive conformation and/or the probability of the polypeptide chemically degrading.

16. The system of claim 14, wherein the actions further comprise ranking the polypeptide lower than another polypeptide in a list of potential polypeptides to be used as at least part of a therapeutic agent based on the probability of the side chain of the amino acid being trapped in the at least one reactive conformation and/or the probability of the polypeptide chemically degrading, wherein the probability of the side chain of the amino acid being trapped in the at least one reactive conformation and/or the probability of the polypeptide chemically degrading for the another polypeptide is less than the probability of the side chain of the amino acid being trapped in the at least one reactive conformation and/or the probability of the polypeptide chemically degrading for the polypeptide.

17. The system of claim 13, wherein the predicting the probability of the polypeptide to chemically degrade includes:
identifying an accessibility constraint that, when satisfied, indicates that an amide group of the polypeptide has above-threshold spatial accessibility to bind with a solvent molecule from a surrounding solvent; and
determining, for the reactive conformation, that the accessibility constraint is satisfied based on assessing one or more spatial characteristics of the polypeptide.

18. The system of claim 11, wherein the determining that the polypeptide conformation is the reactive conformation, comprises:
determining a distance criterion that, when satisfied, indicates that the atom within the side chain is within a predetermined distance threshold of the another atom within the backbone; and
determining that the distance criterion is satisfied for the reactive conformation based on a comparison of the nucleophilic attack distance of the amino acid of the reactive conformation with the predetermined distance threshold.

19. The system of claim 11, wherein the free energy is determined based on analysis of free energy profiles of the dihedral angle for the backbone and the dihedral angle for the side chain of the amino acid in the reactive conformation, and wherein the free energy profiles in spaces of the dihedral angle for the backbone and the dihedral angle for the side chain are calculated from bin populations.

20. A computer-program product tangibly embodied in a non-transitory machine-readable storage medium, including instructions configured to cause one or more data processors to perform actions including:
determining, for a polypeptide conformation of a polypeptide comprising an amino acid having a side chain and a backbone, a dihedral angle for the backbone and a dihedral angle for the side chain of the amino acid while in the polypeptide conformation;
determining a nucleophilic attack distance between two atoms, functional groups, or a combination thereof of the amino acid while in the polypeptide conformation based on the dihedral angle for the backbone and the dihedral angle for the side chain, wherein one of the two atoms or functional groups is in the side chain of the amino acid and another of the two atoms or functional groups is in the backbone of the amino acid;
determining, based on the nucleophilic attack distance of the amino acid while in the polypeptide conformation, that the polypeptide conformation is a reactive conformation that is susceptible to a cleavage reaction;
in response to determining the polypeptide conformation is the reactive conformation, determining a free energy of the dihedral angle for the backbone and the dihedral angle for the side chain of the amino acid while in the reactive conformation; and
predicting a probability of the side chain of the amino acid being trapped in the reactive conformation based on the free energy of the dihedral angle for the backbone and the dihedral angle for the side chain of the amino acid.
